(19) United States
(12) Patent Application Publication
(10) Pub. No.: US 2008/0076972 A1
(43) Pub. Date: Mar. 27, 2008

(54) Integrated sensors for tracking performance metrics

(75) Inventors: Jesse Lee Dorogusker, Los Altos, CA (US); Anthony Fadell, Portola Valley, CA (US); Donald J. Novotney, San Jose, CA (US); Nicholas R. Kalayjian, San Carlos, CA (US)

Correspondence Address: ROPES & GRAY LLP;PATENT DOCKETING 39/361, 1211 AVENUE OF THE AMERICAS, NEW YORK, NY, 10036-8704, US

(73) Assignee: Apple Inc., Cupertino, CA (US)

(21) Appl. No.: 11/729,075

(22) Filed: Mar. 27, 2007

Related U.S. Application Data
(60) Provisional application No. 60/846,414, filed on Sep. 21, 2006.

(51) Int. Cl. A61B 5/00

(52) U.S. Cl. 600/300

(57) ABSTRACT

Electronic devices and accessories having integrated sensors for tracking a user's performance metrics are provided. In one embodiment, the present invention can include a headset having integrated physiological sensors. The present invention also can include a sling having one or more integrated physiological sensors. The sling can secure a portable electronic device to a user's body or clothing. The present invention also can include a portable media device having one or more integrated physiological sensors. In one embodiment, an electronic device of the present invention can accept data from multiple sensors, including one or more sensors that track a user's movements. The electronic device can be configured to condition data from physiological sensors using data indicative of the user's movements.

### CROSS-REFERENCE TO RELATED APPLICATIONS

This is related to:

U.S. Provisional Patent Application No. 60/846,414 to Gilley et al., filed on Sep. 21, 2006 (referred to herein as “the incorporated provisional patent application”), the entirety of which is incorporated herein by reference;

U.S. patent application Ser. No. ______ to Gilley et al., filed on Mar. 27, 2007, entitled “LIFESTYLE COMPANION SYSTEM,” Attorney Docket No. 104677-0019-101/P4725US1 (referred to herein as “the incorporated LIFESTYLE COMPANION document”), the entirety of which is incorporated herein by reference;

U.S. patent application Ser. No. ______ to Gilley et al., filed on Mar. 27, 2007, entitled “SYSTEMS AND METHODS FOR PROVIDING AUDIO AND VISUAL CUES VIA A PORTABLE ELECTRONIC DEVICE,” Attorney Docket No. 104677-0019-102/P4725US2 (referred to herein as “the incorporated AUDIO AND VISUAL CUES document”), the entirety of which is incorporated herein by reference;

U.S. patent application Ser. No. ______ to Gilley et al., filed on Mar. 27, 2007, entitled “DYNAMICALLY ADAPTIVE SCHEDULING SYSTEM,” Attorney Docket No. 104677-0019-103/P4725US3 (referred to herein as “the incorporated ADAPTIVE SCHEDULING SYSTEM document”), the entirety of which is incorporated herein by reference;

U.S. patent application Ser. No. ______ to Gilley et al., filed on Mar. 27, 2007, entitled “SYSTEMS AND METHODS FOR FACILITATING GROUP ACTIVITIES,” Attorney Docket No. 104677-0019-104/P4725US4 (referred to herein as “the incorporated GROUP ACTIVITIES document”), the entirety of which is incorporated herein by reference;

U.S. patent application Ser. No. ______ to Gilley et al., filed on Mar. 27, 2007, entitled “VARIABLE I/O INTERFACE FOR PORTABLE MEDIA DEVICE,” Attorney Docket No. 104677-0025-101/P4732US1 (referred to herein as “the incorporated VARIABLE I/O document”), the entirety of which is incorporated herein by reference; and

U.S. patent application Ser. No. ______ to Gilley et al., filed on Mar. 27, 2007, entitled “REWARDS SYSTEMS,” Attorney Docket No. 104677-0022-101/P4727US1 (referred to herein as “the incorporated REWARDS SYSTEMS document”), the entirety of which is incorporated herein by reference.

### FIELD OF THE INVENTION

The present invention relates to electronic devices and accessories having integrated sensors for tracking a user's performance metrics.

### BACKGROUND OF THE INVENTION

An increasing number of people are exercising to the beat of their favorite songs. Often, the songs are stored on a portable media player strapped to a user's arm so that the user would not need to carry the portable media player during the workout. Audio signals can be transmitted from the portable media player to the user's ear using a set of headphones.

During the workout, the user may want to monitor his performance metrics, e.g., the distance the user has covered during a jog and/or the user's heart rate. Conventionally, this can require the user to attach additional electronic devices to his body or clothing. For example, to monitor the distance a user has jogged, the user may clip a pedometer to his clothing. To obtain information about the user's heart rate, the user may strap a heart rate sensor to his chest and a receiver for receiving data from the heart rate sensor to his wrist. Disadvantageously, the user can become very uncomfortable during his fitness routine due to the number of devices the user has attached to his body and clothing.

### SUMMARY OF THE INVENTION

The present invention can include systems and methods for integrating sensors for tracking a user's performance metrics into electronic devices and accessories therefor. In one embodiment of the present invention, the electronic devices can include portable media devices similar to iPods™ sold by Apple Inc. of Cupertino, Calif. or cellular telephones similar to iPhones™ designed by Apple Inc.

In one embodiment, the present invention can include headsets having one or more integrated physiological sensors. The headsets can be, for example, headphones for receiving audio signals from a media device or headsets for communication with cellular telephones. The sensor signals can be transmitted to a data repository through the same communication channel through which audio signals are transmitted to the headset (e.g., hard-wired or wireless).

In one embodiment, the present invention can include a sling having one or more integrated physiological sensors. The sling can have an enclosure within which a portable electronic device can be disposed. The sling also can have a strap-for securing the sling and the portable electronic device to the user's body or clothing. In one embodiment of the present invention, the sling can have an integrated connector configured to be operatively coupled to a complementary connector in the portable electronic device. The sensor signals can be transmitted from the sling to the portable electronic device via these connectors. Alternatively, the sensor signals can be transmitted via wireless communication protocols.

In one embodiment, the present invention can include a portable media device having one or more integrated physiological sensors. The physiological sensors can (1) be exposed through the housing of the portable media device, or (2) be hidden from view and undetectable by the user. A sling for securing such a portable electronic device to the user's body also is provided by the present invention.

In one embodiment of the present invention, an electronic device can accept data from one or more physiological sensors, along with data from one or more other sensors that track the user's movements. The movement sensors can be disposed within the electronic device itself or attached to or incorporated within the user's body or clothing (e.g., a movement sensor similar to the sensor from the Nike+ipod Sport Kit sold by Apple Inc. and Nike Inc. of Beaverton, Oreg.). The electronic device can be configured to condition the data from the physiological sensors using the data from the movement sensors. For example, because the physiological sensor may contain anomalies due to the user's movements, the electronic device can identify the anomalies based on data indicative of the user's movements and thereafter remove or filter out their effects.

### BRIEF DESCRIPTION OF THE DRAWINGS

The above and other advantages of the present invention will be apparent upon consideration of the following detailed description, taken in conjunction with accompanying drawings, in which like reference characters refer to like parts throughout, and in which:

FIG. 1A illustrates a prior art portable media system;

FIG. 1B illustrates a prior art physiological monitoring system;

FIG. 2 illustrates an earbud of a headset having one or more integrated physiological sensors in accordance with one embodiment of the present invention;

FIGS. 3A-3B illustrate an alternative earbud of a headset having one or more integrated physiological sensors in accordance with one embodiment of the present invention;

FIG. 4 shows a third illustrative headset having one or more integrated physiological sensors and an illustrative portable electronic device in accordance with one embodiment of the present invention;

FIG. 5 illustrates an adapter for providing hard-wired communication between a headset and an electronic device in accordance with one embodiment of the present invention;

FIGS. 6A-6B illustrate an armband having one or more integrated physiological sensors in accordance with one embodiment of the present invention;

FIGS. 7A-7B illustrate a portable electronic device having one or more integrated physiological sensors in accordance with one embodiment of the present invention;

FIG. 7C illustrates an armband for use with the portable electronic device of FIGS. 7A-7B in accordance with one embodiment of the present invention;

FIG. 8 illustrates a portable electronic device that accepts data from multiple sensors in accordance with one embodiment of the present invention; and

FIGS. 9A-9C illustrate signal conditioning of data from a physiological sensor using data indicative of a user's movements in accordance with one embodiment of the present invention.

### DETAILED DESCRIPTION OF THE INVENTION

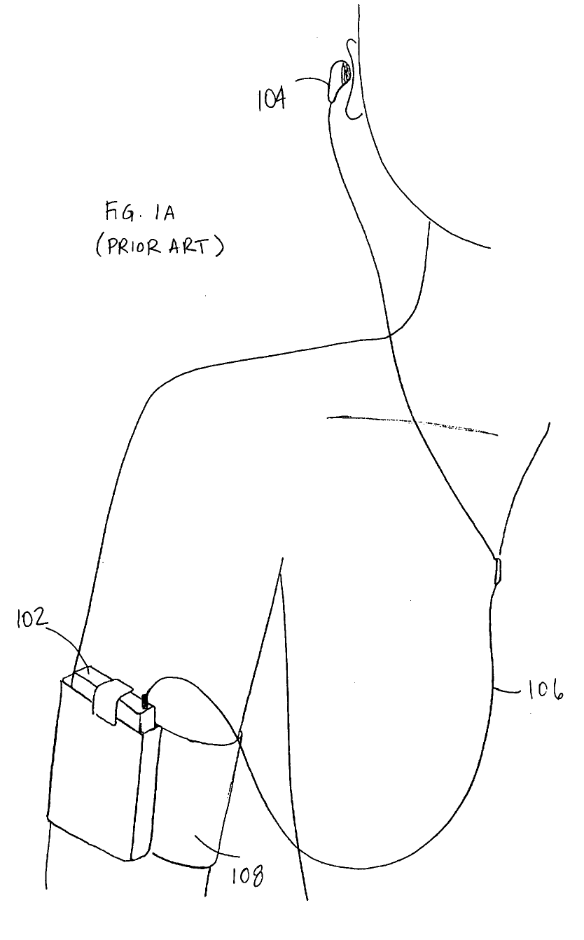

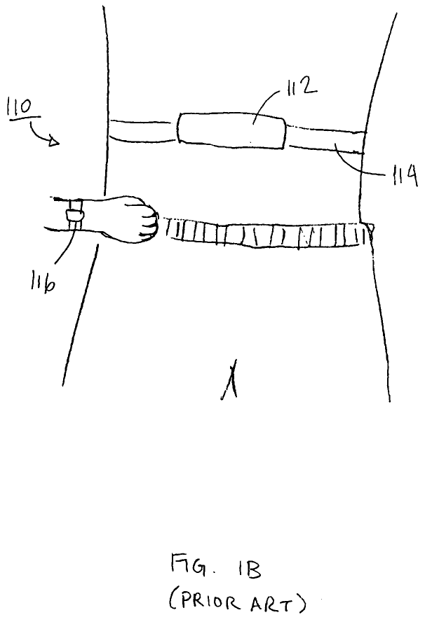

FIG. 1A illustrates a prior art portable media system having portable media device 102, headset 104, and armband 106. Portable media device 102 can provide audio signals to a user via headset 104. The audio signals can be transmitted to headset 104 using wires 106. While the user is exercising or otherwise does not wish to carry portable media device 102 in his hands, the user can strap the portable media device to his arm using armband 108.

FIG. 1B illustrates a prior art physiological monitoring system. A user may use conventional heart rate monitoring system 110 to monitor the user's heart rate, e.g., while the user is performing an exercise routine. Conventional heart rate monitoring system 110 can be equipped with heart rate sensor 112, which the user can strap onto the user's chest using chest band 114. Heart rate monitoring system 110 also can have receiver 116, which can accept wireless data signals transmitted from heart rate sensor 112 and display such information for the user's consideration. Typically, receiver 116 is configured to be strapped onto a user's wrist in a manner similar to that of a watch.

So, if a user wants to listen to be entertained, e.g., by audio tracks stored on portable media device 102 and simultaneously monitor his performance metrics during a workout, the user would need to strap his portable media player onto his arm, a heart rate sensor to his chest, and a receiver to his wrist. Unfortunately, due to the numerous electronic devices strapped to the user, the user may experience considerable discomfort.

The present invention can include systems and methods for integrating sensors for tracking a user's performance metrics into media devices and accessories therefor, thereby reducing or eliminating the need for additional independent monitoring devices. Advantageously, such integration of sensors can be desirable because it requires no additional effort by the user to use them. As used herein, performance metrics can include physiological metrics (e.g., heart rate, EKG, blood oxygen content, temperature, heat flux, etc.) and non-physiological indications of performance (e.g., distance covered, pace, etc.).

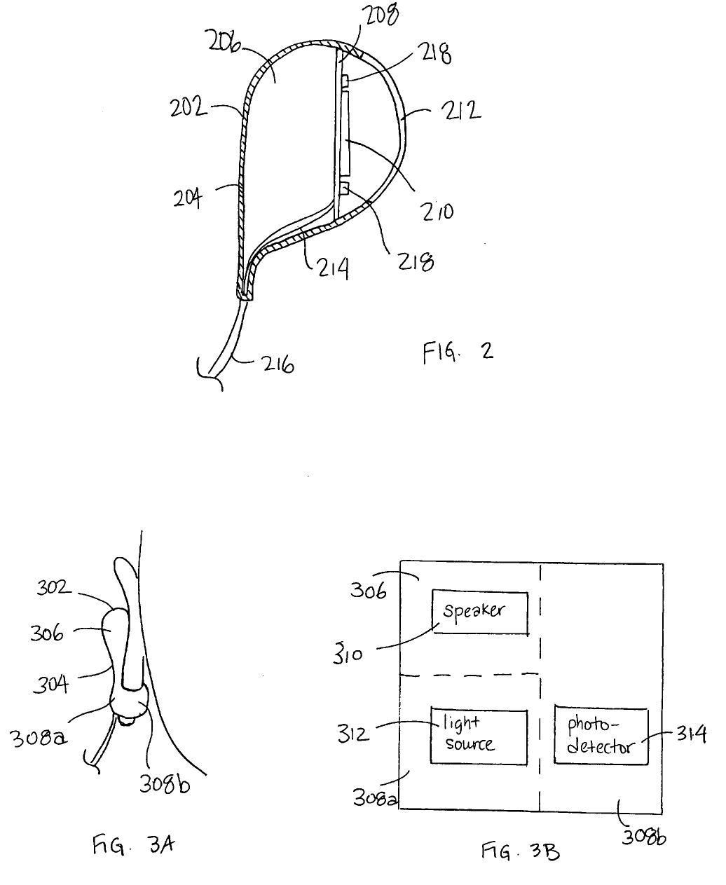

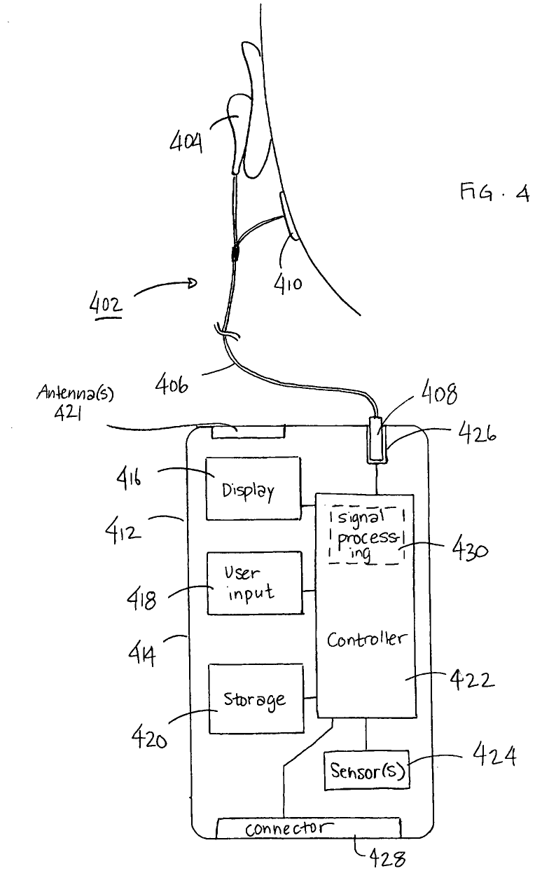

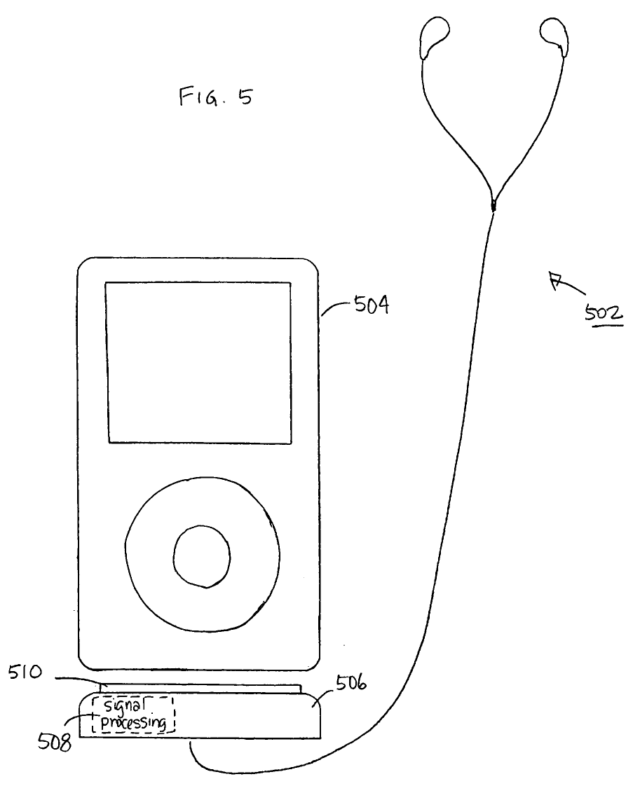

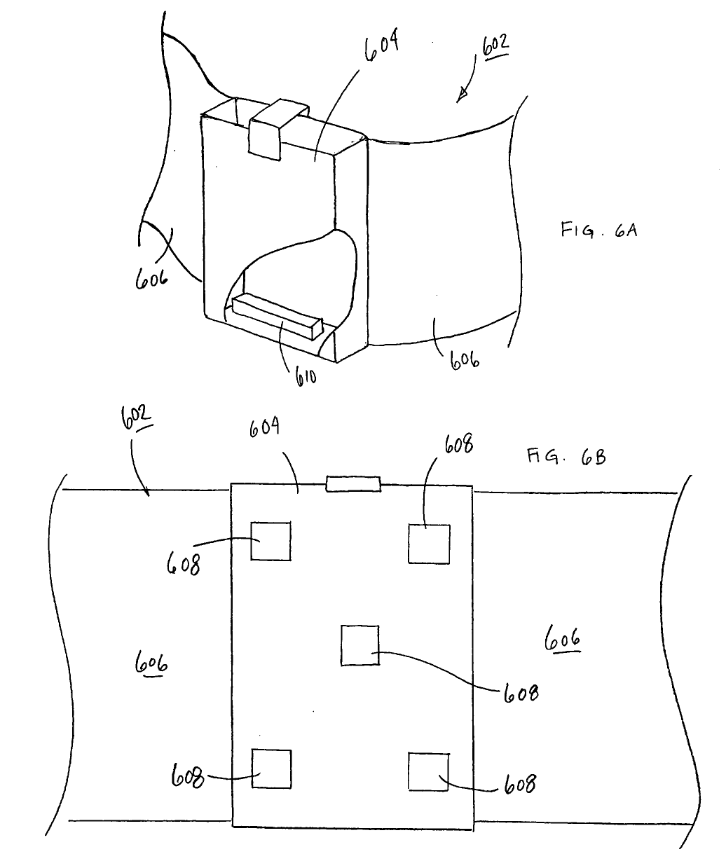

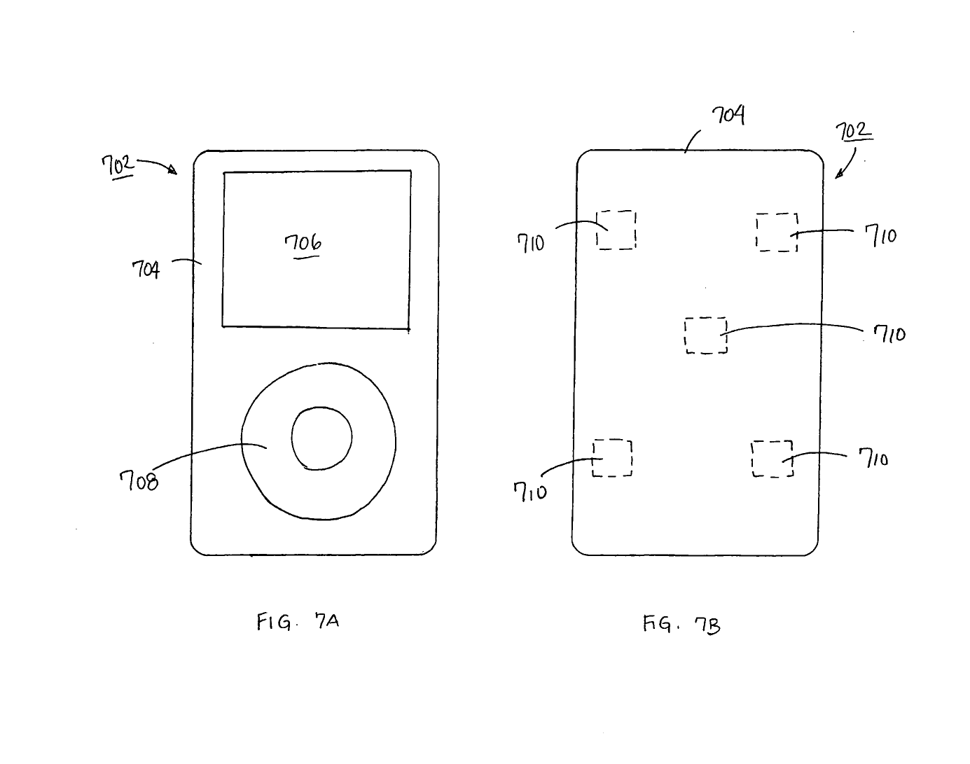

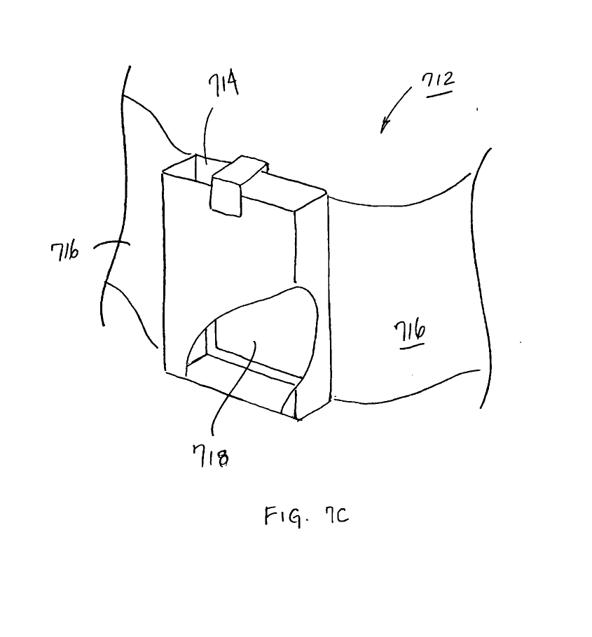

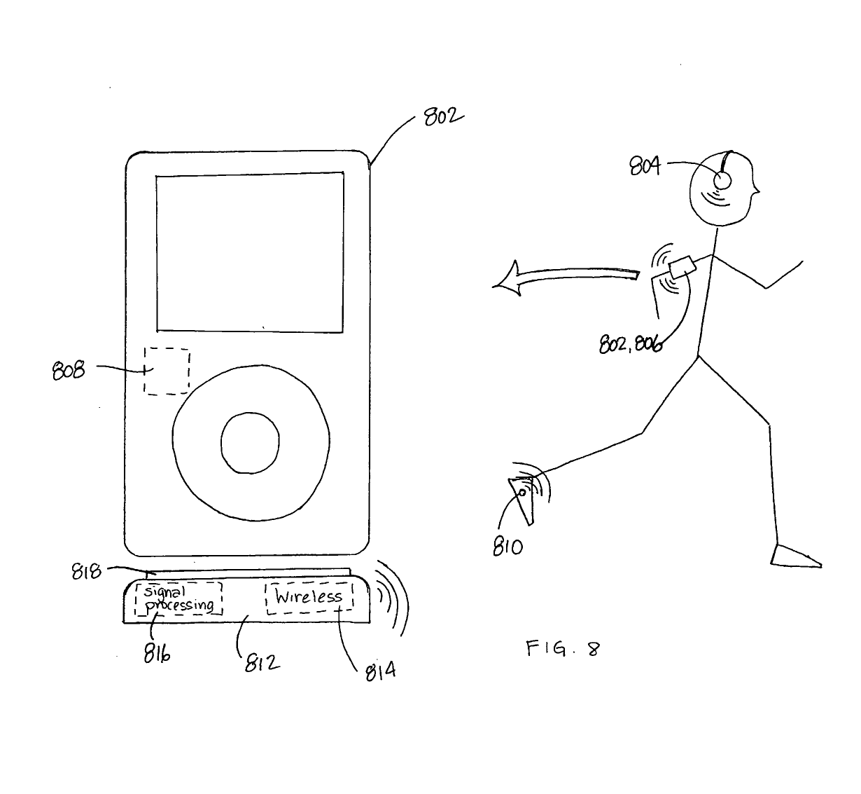

FIG. 2 illustrates an earbud of a headset having one or more integrated physiological sensors in accordance with one embodiment of the present invention. Earbud 202 can have housing 204 and internal cavity 206. Internal cavity 206 can be bisected by internal wall 208 into separate acoustical chambers. Speaker 210 can be positioned within internal cavity 206 (e.g., mounted onto internal wall 208) so that sound waves emanating from the speaker are directed out of acoustical aperture 212. Speaker 210 can be coupled to wires 214 that transmit audio signals from an electronic device (not shown). Outside earbud 202, wires 214 can be encased in a non-conductive material to form cable 216. One embodiment of the electronic device of the present invention is described in greater detail below with respect to FIG. 4.

In one embodiment of the present invention, earbud 202 also can be equipped with one or more physiological sensors 218. For example, sensors 218 can include one or more infrared photodetectors for tracking the user's temperature, heat flux, and heart rate. From infrared radiation in the user's ear, sensors 218 can detect minute temperature variations due to the user's heart beats. Heart rate can be calculated based on the time between beats and the user's temperature can be set as the “DC component” (or average or median value) of the detected temperature distribution. Other sensors also can be used for tracking the same physiological metrics or different physiological metrics. In one embodiment of the present invention, one sensor 218 can be centered with respect to acoustical aperture 212 to ensure that the sensor receives a sufficient infrared signal regardless of how the user positions earbud 202 in the user's ear. Alternatively, speaker 210 can be centered with respect to acoustical aperture 212 and two or more of the same sensors 218 can be positioned around the periphery of speaker 210. This can increase the likelihood that at least one sensor 218 can obtain a sufficient infrared signal regardless of how the user positions earbud 202 in the user's ear.

Sensors 218 can send data signals to a signal processor. The signal processor can be disposed in earbud 202, in an electronic device from which the earbud receives audio signals, and/or in an adapter configured for coupling to the electronic device. Illustrative embodiments of an electronic device and adapter are described in greater detail herein.

In accordance with one aspect of the present invention, earbud 202 can be incorporated into a wired or wireless headset that receives and/or transmits audio signals. For example, if the electronic device with which the headset communicates is a telephone, the headset also can be equipped with a microphone. The telephone can be based on any one of the following networks: public switched telephone network (PSTN), cellular, voice over internet protocol (VoIP), any other telephone network, or any combination thereof. Each earbud in the headset can have sensors 218 or only one of the earbuds can have sensors 218.

FIGS. 3A-3B illustrate an alternative earbud of a headset having one or more integrated physiological sensors in accordance with one embodiment of the present invention. Earbud 302 can have housing 304 with main body portion 306 and clip portions 308a and 308b. Main body portion 306 can be equipped with speaker 310 for delivering acoustic signals to the user and, in some embodiments, physiological sensors similar to sensors 218 of FIG. 2. When main body portion 306 is disposed in a user's ear, clip portion 308a can be configured to be disposed near or against the front lobe of the user's earlobe, and clip portion 308b can be configured to be disposed near or against the back lobe of the user's earlobe (or vice versa). In one embodiment of the present invention, a spring force can be provided to bias clip portions 308a and 308b against the user's earlobe.

Light source 312 can be disposed within clip portion 308a or 308b, and photodetector 314 can be disposed within the other clip portion. Clip portions 308a and 308b can have apertures through which light can be transmitted and received by light source 312 and photodetector 314, respectively. Alternatively, clip portions 308a and 308b can be made at least partially of a transmissive material through which the light can pass.

In one embodiment of the present invention, light source 312 and photodetector 314 can be used to track a user's blood oxygen content and heart-rate. For example, light source 312 can include two light emitting diodes that provide light at two different wavelengths. Photodetector 314 can detect the portion of such light that passes through the user's earlobe, the remaining light having been absorbed by the blood within the user's earlobe. From the relative absorption of these two wavelengths of light, the user's blood oxygen content can be calculated. The user's heart beat also can be determined based on the photodetector signal since the signal may bounce in time with the user's heart beat due to the expansion and contraction of arterial blood vessels. In an alternative embodiment, both light source 312 and photodetector 314 can be disposed in either clip portion 308a or 308b and operate as a reflectance pulse oximeter.

Light source 312 can receive signals from and photodetector 314 can send data signals to a controller that controls the light source and photodetector and processes signals sent thereto or received therefrom. The controller can be disposed in earbud 302, in an electronic device from which the earbud receives audio signals, and/or in an adapter configured for coupling to the electronic device.

In accordance with an alternative embodiment of the present invention, electrical contacts (not shown) also can be disposed within clip portion 308a and/or 308b, in addition to or instead of light source 312 and photodetector 314. Electrical contacts can include contacts for detecting, e.g., temperature, heat flux, EKG waveforms, heart rate, any other suitable physiological metric, or any combination thereof. For example, to detect EKG waveforms, each earbud of a headset can be equipped with electrical contacts. The placement of the electrical contacts in both the left and right hemispheres of the user body and the resulting distance between electrical contacts can permit the present invention to detect greater voltage differences, and thus more accurate EKG data. The user's heart rate can be extracted from the EKG waveforms, although, in some embodiments of the present invention, heart rate can be determined without detecting EKG waveforms.

FIG. 4 shows a third illustrative headset having one or more integrated physiological sensors and an illustrative portable electronic device in accordance with one embodiment of the present invention. Headset 402 can incorporate earbud 404, cable 406, audio connector 408, and sensor 410. Earbud 404 can be equipped with a speaker and, in some embodiments, sensors similar to those described with respect to FIGS. 2 and 3A-3B. Cable 406 can electrically and mechanically couple earbud 404 and sensor 410 to audio connector 408. Sensor 410 can be any one of the sensors described herein for tracking a user's physiological metrics. For example, sensor 410 can include electrical contacts for measuring heart rate or sensors for a reflectance pulse oximeter.

To promote contact between sensor 410 and the user's skin, the sensor can be partially or fully encased in a material that causes it to be removably attached to the user's skin. For example, sensor 410 can be encased in a material that is attracted to the ionic charges on the user's skin.

Portable electronic device 412 can have housing 414 within which is disposed display 416, user input component 418, memory 420, antenna(s) 421, controller 422, sensors 424, audio connector receptacle 426, and connector 428.

User input component 418 can permit a user to interact with the electronic device. User input component 418 can include a clickwheel similar to that incorporated in some models of iPods™. The clickwheel can include one or more buttons and a touchpad. The touchpad can permit a user to scroll by running the user's finger around the track of the clickwheel. In alternative embodiments, user input component 418 can include, for example, one or more buttons, a touchpad, a touchscreen display, electronics for accepting voice commands, antennas for accepting signals from other electronic devices, infrared ports for accepting signals from other electronic devices, or any combination thereof. In one embodiment of the present invention, display 416 and user input 418 can be integrated into one component by using a touchscreen display.

Memory 420 can store sensor data generated by sensor 410, sensor(s) 424, and/or any other sensor used for tracking a user's performance metrics. Memory 420 can include read only memory, random access memory, solid-state memory, buffer memory, hard drive memory, any other memory known in the art or otherwise, or any combination thereof.

In one embodiment of the present invention, memory 420 also can store media files for playback by electronic device 412. Media files can include, for example, audio files for playback through speakers (e.g., headset 402) and/or graphic images for playback on a display (e.g., display 416). The media files can include media files that a user has selected to be played back during performance of an activity to entertain and motivate the user. Such media files can include, for example, songs, audio books, multimedia presentations, still images, text, podcasts, videos, etc.

Electronic device 412 can have one or more antennas 421 for wirelessly communicating with a data network and/or with one or more accessories (e.g., any of the headsets described herein). For example, the electronic device can include one or more antennas for communication with Bluetooth-enabled devices, WiFi network, cellular network, radio network, or any combination thereof. In one embodiment of the present invention, antenna(s) 421 can permit a user to stream or otherwise download audio and/or visual media to entertain and motivate the user during performance of an activity. For example, the user may choose to stream internet radio or a podcast through a WiFi network. If electronic device 412 is a cellular telephone, the user may choose to have a telephone conversation with another person by connecting to a cellular network.

Controller 422 can, for example, control operation of electronic device 412, generate audio signals for transmission to a headset, instruct audio signals to be transmitted to a headset, generate control signals for any of the sensors described herein, and/or accept data signals from any of the sensors described herein. Controller 422 can include one or more processors, ASICs, circuits, or any combination thereof.

Display 416 can provide graphics (e.g., text, still images, and/or videos) to the user. In one embodiment of the present invention, display 416 can provide the user with graphics about the user's performance metrics. Display 416 also can provide entertainment graphics from media files stored in memory 420 or downloaded from a data network via antenna(s) 421. In one embodiment of the present invention, controller 422 can instruct display 416 to display graphics about the user's performance metrics and entertainment graphics simultaneously. Advantageously, this permits a user to monitor his performance metrics while simultaneously be entertained during his workout.

Sensors 424 can incorporate sensors described in greater detail with respect to FIGS. 7A-7B and/or other sensors that can track the user's performance metrics. In one embodiment of the present invention, sensor 424 can be a sensor for tracking a user's movements. In one embodiment, movement sensors can include an accelerometer or GPS chip for detecting a user's movements while walking or jogging.

Data from sensors 424 and any other sensor for tracking performance metrics can be transmitted to signal processing module 430 of controller 422 for signal processing. For example, signal processing module 430 can incorporate digital circuitry, analog circuitry, and/or software for amplifying and filtering the sensor data.

Audio connector receptacle 426 can be configured to accept audio connector 408 of headset 402. In one embodiment of the present invention, audio connector 408 and receptacle 426 can transmit control signals to and accept data from sensors integrated with headset via a dedicated electrical lead in audio connector 408. Alternatively, the control signals and sensor data can be interleaved with other signals using, e.g., a serial interface protocol.

In a further alternative embodiment, the control signals and sensor data can be transmitted through connector 428, as illustratively described in greater detail with respect to FIGS. 5 and 8. In one embodiment of the present invention, connector 428 can be a multiple pin connector similar to that described in U.S. Patent Application Publication No. 20050240705 to Novotney et al., published on Oct. 27, 2005, the entirety of which is incorporated by reference herein. Electronic device 412 can use connector 428 for data communication with a local server, e.g., the user's personal computer.

In one embodiment of the present invention, controller 422 can generate audio signals related to the user's performance metrics. These audio signals can be intermixed with audio signals from entertainment media files stored in memory 420 or audio signals downloaded by the user from data networks (e.g., the internet or radio broadcast).

In one embodiment of the present invention, electronic device 412 can be an iPod™ or an iPhone™. Electronic device 412 also can be any electronic device suitable for processing signals from sensors that track a user's performance metrics during physical activity. For example, the electronic device can be any portable, mobile, hand-held, or miniature consumer electronic device. Illustrative electronic devices can include, but are not limited to, music players, video players, still image players, game players, other media players, music recorders, video recorders, cameras, other media recorders, radios, medical equipment, calculators, cellular phones, other wireless communication devices, personal digital assistants, programmable remote controls, pagers, laptop computers, printers, or any combination thereof. Miniature electronic devices may have a form factor that is smaller than that of hand-held devices. Illustrative miniature electronic devices can include, but are not limited to, watches, rings, necklaces, belts, accessories for belts, headsets, accessories for shoes, virtual reality devices, other wearable electronics, accessories for sporting equipment, accessories for fitness equipment, key chains, or any combination thereof.

While electronic device 412 is illustratively shown in operation with headset 402 in FIG. 4, electronic device 412 can be used alone or in combination with any of the accessories described herein.

FIG. 5 illustrates-an adapter for providing hard-wired communication between a headset and an electronic device in accordance with one embodiment of the present invention. Headset 502 can be similar to any of the headsets described herein, e.g., the headsets described with respect to FIGS. 2, 3A-3B, and 4. Headset 502 can be coupled to electronic device 504 via adapter 506. Adapter 506 can have connector 510 configured for electrical and mechanical coupling to a complementary connector in electronic device 504 (e.g., a connector similar to connector 428 of FIG. 4).

In one embodiment of the present invention, adapter 506 can incorporate signal processing module 508 for processing signals from sensors integrated with headset 502, in contrast to the embodiment of FIG. 4 which illustratively can integrate a similar signal processing module within electronic device 412. During operation, a controller within electronic device 504 can transmit audio and control signals for headset 502 via adapter 506. Sensor data also can be transmitted back to electronic device 504 via adapter 506, after having been conditioned by signal processing module 508 in adapter 506. In one embodiment of the present invention, connector 510 can transmit audio and control signals to and accept data from sensors integrated with headset via dedicated electrical lead(s) in the connector. Alternatively, the audio and control signals and sensor data can be interleaved with other signals transmitted through connector 510.

FIGS. 6A-6B illustrate an armband having one or more integrated physiological sensors in accordance with one embodiment of the present invention. Armband 602 can have enclosure 604 within which a portable electronic device similar to, e.g., electronic device 412, can be disposed. To secure enclosure 604 (and thus any electronic device disposed therein) to a user's arm, armband 602 can have strap 606, e.g., an elastic strap with Velcro.

In one embodiment of the present invention, armband 602 can be equipped with one or more integrated sensors 608 for tracking the physiological metrics of the user. Sensors 608 can be integrated into, e.g., a back wall of enclosure 604 or within strap 606. When armband 602 is strapped onto a user's arm, the sensors can be positioned in close proximity to or in contact with the user's skin.

One or more sensors 608 can be electrical contacts for tracking, e.g., a user's EKG waveforms, heart rate, temperature, and/or heat flux. The electrical contacts can be exposed to the external environment or obtain physiological signals through material that is conducive to electrical and/or thermal conduction. For example, one or more sensors 608 can include electrical contacts similar to those described above with respect to FIG. 4.

One or more sensors 608 also can include an optical sensor system for tracking, e.g., the user's oxygen content, heart rate, temperature, and/or heat flux. The optical sensor system can transmit and/or receive light through an aperture in a wall of the armband or through a light-transmissive material. For example, one or more sensors 608 can include a light source and photodetector similar to those described above with respect to FIGS. 3A-3B, or an infrared photodetector similar to that described above with respect to FIG. 2.

To transmit sensor data to an electronic device for additional processing, analysis, and/or storage, armband 602 can have an integrated connector 610 (as shown in the cut-away view of FIG. 6A). Connector 610 can be configured for electrical and mechanical coupling to a complementary connector of the electronic device (e.g., connector 428 of FIG. 4). Wires integrated within the armband can operatively couple sensors 608 to connector 610. Alternatively, rather than having a connector for hard-wired communication between sensors 608 and an electronic device, the sling can instead be equipped with a wireless transmitter and the electronic device can be equipped with a wireless receiver (either integrated within the device or via an adapter as discussed in greater detail with respect to FIG. 8).

In alternative embodiments of the present invention, sensors 608 and connector 610 also can be integrated in other types of slings. For example, the sensors and connector also can be integrated into leg-bands and chest-bands.

FIGS. 7A-7B illustrate a portable electronic device having one or more integrated physiological sensors in accordance with one embodiment of the present invention. Electronic device 702 can have housing 704, display 706, and user input component 708. Electronic device 702 also can have one or more integrated sensors 710 for tracking the user's physiological metrics. For example, one or more sensors 710 can be similar to sensors 608 of FIG. 6B. In one embodiment of the present invention, one or more sensors 710 can be disposed to interact with the user from a back face of the electronic device. Alternatively, one or more sensors 710 can be disposed along a side surface of the electronic device. When disposed on a side surface of the electronic device, the sensors can interact with the user when the user is holding the electronic device along its side surfaces. In one embodiment, one or more sensors can be disposed underneath an external surface of the electronic device, as indicated by the dashed lines. For example, if one or more sensors are IR sensors, the external surface separating the sensors from the external environment can be made from a light-transmissive material. If the sensors are electrical contacts (e.g., for detecting heart rate, EKG waveforms, temperature, or heat flux), external surface separating the sensors from the external environment can be made from material that is conducive to electrical and/or thermal conduction.

Electronic device 702 also can have additional electronic components similar to those described with respect to electronic device 412 of FIG. 4 (e.g., memory, a controller, a connector, antennas, and sensors).

FIG. 7C illustrates an armband for use with the portable electronic device of FIGS. 7A-7B in accordance with one embodiment of the present invention. Armband 712 can have enclosure 714 and strap 716 for securing enclosure 714 and any electronic device disposed therein to the user's body. Armband 712 also can have cutout 718 to permit sensors 710 of electronic device 702 to be disposed closer to or in contact with the user's skin when the armband is strapped to the user.

FIG. 8 illustrates a portable electronic device that accepts data from multiple sensors in accordance with one embodiment of the present invention. Electronic device 802 can accept data from one or more physiological sensors, along with data from one or more other sensors that track the user's movements. The physiological sensors can be integrated into, e.g., the portable electronic device itself and/or one or more accessories for the electronic device. The physiological sensors also can be disposed within the user's clothing or otherwise attached to the user's body. Sensors for tracking the user's movements can be disposed within the electronic device itself (e.g., an accelerometer or GPS sensor 808), attached to or incorporated within the user's clothing or body (e.g., a sensor similar to the sensor from the Nike+ipod Sport Kit sold by Apple Inc. and Nike Inc. of Beaverton, Oreg.), or integrated in other devices or products used by the user during performance of an activity (e.g., within exercise equipment).

In one embodiment of the present invention, adapter 812 having wireless communication module 814 can be provided to facilitate wireless communication between electronic device 802 and one or more accessories therefor (e.g., headset 804, sensor 810, and/or armband 806). For example, adapter 812 can be configured to communicate with headset 804 using the Bluetooth protocol and with sensor 810 using another radio-frequency wireless communication protocol.

Adapter 812 also permits electronic device 802 to receive sensor data from multiple sensors for tracking performance metrics, including, e.g., sensor(s) in headset 804 and sensor 810. Upon receipt, the sensor data can be routed to signal processing module 816 disposed within adapter 818 or within electronic device 802. The signals can be transmitted via connector 818 and a complementary connector in electronic device 802 to a controller within the electronic device for additional conditioning, analysis, or storage. Adapter 812 also can transmit audio and control signals generated by electronic device 802 to accessories, e.g., headset 804.

In an alternative embodiment of the present invention, one or more components of adapter 812 can be disposed within electronic device 802. For example, adapter 812 can be eliminated by equipping electronic device 802 with all of the components disposed in adapter 812. Thus, electronic device 802 can wirelessly communicate with headset 804, sensor 810, and/or armband 806 directly. Electronic device 802 also can be configured to communicate directly with multiple accessories and data sources (without use of adapter 812) using a combination of hard-wired and wireless connections.

Electronic device 802 also can be configured to communicate with multiple accessories and data sources using only hard-wired connections, either indirectly (e.g., through an adapter) or directly (e.g., without an adapter). For example, a hard-wired adapter can have multiple receptacles and a multiplexer for facilitating receipt of data from multiple sources. The hard-wired adapter can communicate with electronic device 802 using a connector similar to connector 818.

Advantageously, by being able to accept sensor data from multiple sources, the electronic devices of the present invention can correlate data from the multiple sources, analyze each data set with respect to one or more other data sets, and provide the user with a more comprehensive picture of the user's workout. For example, in one embodiment of the present invention, an electronic device of the present invention can be configured to condition the data from one or more physiological sensors using data indicative of the user's movements (e.g., from sensor 810).

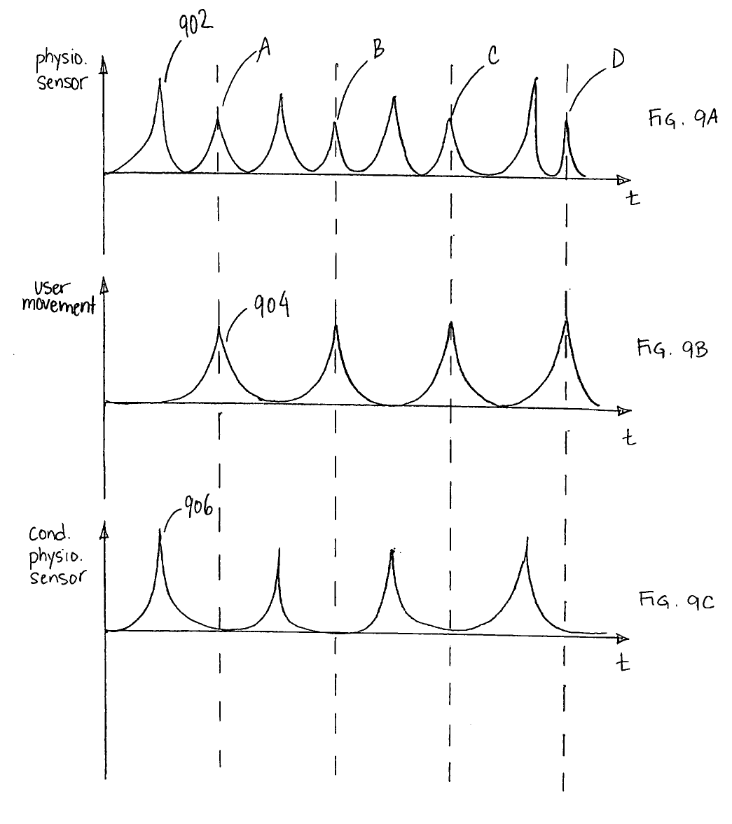

FIGS. 9A-9C illustrate signal conditioning of data from a physiological sensor using data indicative of a user's movements in accordance with one embodiment of the present invention. FIG. 9A shows illustrative waveform 902 generated by signals from a physiological sensor of the present invention. Unfortunately, due to the sensitivity of some physiological sensors, waveform 902 may contain anomalies resulting from the user's movements. An electronic device of the present invention can be configured to identify the anomalies based on data indicative of the user's movements and thereafter remove or filter out their effects. For example, physiological sensor waveform 902 can incorporate anomalies at points A-D. As shown in FIG. 9B, these anomalies can correspond and relate to data points in waveform 904, which can be generated by signals from a sensor that tracks a user's movements (e.g., sensor 810 of FIG. 8). The present invention can be configured to identify the anomalies in waveform 902 based on data from waveform 904 and thereafter remove or filter out their effects, as shown in waveform 906 of FIG. 9C.

Although particular embodiments of the present invention have been described above in detail, it will be understood that this description is merely for purposes of illustration. Alternative embodiments of those described hereinabove also are within the scope of the present invention.

Combinations of the above-described embodiments of the present invention or portions thereof may be provided in one system without departing from the scope of the present invention. For example, earbuds 202 and 302 of FIGS. 2 and 3A-3B can be combined with sensor 410 of FIG. 4. Alternatively, armband 602 of FIG. 6 can be combined with any of the headset and/or electronic devices described herein.

While the description herein may focus on particular sensors for tracking a user's performance metrics, the present invention can employ any type of sensors, including, e.g., pedometers, accelerometers, pressure sensitive sensors, strain sensors, contact switches, heart rate monitors, oximeters, location-tracking (e.g., GPS) sensors, temperature sensors, heat flux sensors, electrocardiogram sensors, scales, glucometers, any other suitable sensor useful for capturing data about a person's performance metrics, or any combination thereof. Any of the physiological sensors described herein with respect to FIGS. 1-8 can be replaced or augmented with movement sensors or any other sensors that track performance metrics.

While the description herein may focus on the use of the present invention in tracking a user's performance metrics during a workout, the present invention also can be used to track a user's performance metrics during any other physical or mental activity. For example, the present invention can be used to monitor how a user's heart rate, EKG waveforms, temperature, and blood oxygen levels respond to stress, e.g., when the user is taking an exam.

The above described embodiments of the present invention are presented for purposes of illustration and not of limitation, and the present invention is limited only by the claims which follow.

What is claimed is:

1. A media system comprising:
a headset having at least a first earbud, wherein the first earbud has a housing;
a speaker disposed in the first earbud;
an acoustic aperture for delivering sound waves from the speaker to an external environment, wherein the acoustic aperture is disposed through the housing of the first earbud; and
at least a first physiological sensor for tracking at least a first physiological metric, wherein the first physiological sensor is integrated with the headset.

2. The media system of claim 1, further comprising a wireless communication module disposed in the headset to facilitate communication of data from the first physiological sensor.

3. The media system of claim 2, wherein the wireless communication module comprises a Bluetooth antenna.

4. The media system of claim 1, wherein the first physiological sensor comprises an optical sensor system.

5. The media system of claim 1, wherein the first physiological sensor comprises one or more electrical contacts.

6. The media system of claim 1, wherein data generated by the first physiological sensor provides information related to at least two physiological metrics.

7. The media system of claim 1, further comprising a portable media player having memory for storing data from the first physiological sensor and for storing entertainment media files.

8. The media system of claim 7, wherein the portable media player further comprises a media output for transmitting output signals from at least one of the entertainment media files.

9. The media system of claim 8, wherein the media output is configured to accept data generated by the first physiological sensor.

10. The media system of claim 8, wherein the media output is configured to transmit signals wirelessly.

11. An accessory for a portable media device, the accessory comprising:
an enclosure within which the portable media device can be disposed;
a strap attached to the enclosure for securing the enclosure to a user's body;
at least a first physiological sensor for tracking at least a first physiological metric, wherein the first physiological sensor is integrated with the enclosure or the strap; and
a communication module for transmitting signals from the first physiological sensor to the portable media device.

12. The accessory of claim 11, wherein the communication module comprises a connector.

13. The accessory of claim 11, wherein the communication module comprises a wireless communication module.

14. The accessory of claim of claim 11, wherein the first physiological sensor is exposed to the external environment.

15. The accessory of claim of claim 11, wherein a light transmissive material shields the first physiological sensor from the external environment.

16. The media system of claim 11, wherein the optical sensor system comprises a photodetector.

17. The media system of claim 16, wherein the optical sensor system comprises a light source.

18. The media system of claim 11, wherein the first physiological sensor comprises one or more electrical contacts.

19. A portable media system comprising:
a portable media device;
memory for storing media files and physiological sensor data, wherein the memory is disposed within the portable media device and wherein the physiological sensor data represents at least a first physiological metric;
a media output disposed within the portable media device for transmitting output signals from at least one of the media files; and
at least a first physiological sensor for generating the physiological sensor data, wherein the first physiological sensor is integrated with the portable media device.

20. The portable media system of claim 19, further comprising a controller, wherein the media output comprises a display and the controller is configured to instruct the display to show entertainment graphics from media files stored in the memory.

21. The portable media system of claim 20, wherein the controller is configured to instruct the display to show graphics related to the first physiological metric simultaneously with the entertainment graphics.

22. The portable media system of claim 19, further comprising a controller, wherein the media output comprises an audio output connector and the controller is configured to output audio signals from at least one of the media files through the audio output connector.

23. The portable media system of claim 19, further comprising a headset having a speaker, a media input, and at least a second physiological sensor for generating physiological sensor data, wherein the media input of the headset is configured to be compatible with the media output of the portable media device and the media input is configured to transmit data from the second physiological sensor.

24. The portable media system of claim 23, wherein the media output of the portable media device comprises a first wireless communication module and the media input of the headset comprises a second wireless communication module, wherein data generated by the first physiological sensor is transmitted from the headset to the portable media player via the first and second wireless communication modules.

25. A method for a media device to provide entertainment to a user and track the user's performance metrics simultaneously, the method comprising:
generating audio signals from entertainment media files stored in memory;
communicating the audio signals to an electronic accessory via a communications channel; and
accepting data generated by a physiological sensor disposed in the electronic accessory via the communications channel.

26. The method of claim 25, further comprising:
accepting data generated by a movement sensor; and
conditioning data generated by the physiological sensor using data generated by the movement sensor.

27. The method of claim 26, further comprising:
storing data generated by the physiological sensor;
accepting data generated by a movement sensor; and
storing data generated by the movement sensor.

28. The method of claim 25, further comprising:
generating graphic signals from entertainment media files stored in memory; and
communicating the graphic signals to a display.

29. A method for an electronic accessory to provide entertainment to a user and track the user's performance metrics simultaneously, the method comprising:
receiving audio signals from a media device via a communications channel;
driving a speaker using the audio signals;
using a physiological sensor in the electronic accessory to generate physiological data; and
transmitting the physiological data from the electronic accessory via the communications channel.

30. The method of claim 29, wherein the communications channel is a wireless communications channel.

31. The method of claim 29, wherein using a physiological sensor in the electronic accessory to generate physiological data comprises using an optical sensor system to generate physiological data.

32. The method of claim 29, wherein using a physiological sensor in the electronic accessory to generate physiological data comprises using a physiological sensor in the electronic accessory to generate physiological data that provides information related to at least two physiological metrics.
